(19) United States
(12) Patent Application Publication
(10) Pub. No.: US 2004/0127856 A1
(43) Pub. Date: Jul. 1, 2004

(54) Catheter and delivery end

(76) Inventor: Carolyn Johnson, St. Peters, MO (US)

Correspondence Address: Carolyn Johnson, 10 Fawn Lake Dr., St. Peters, MO, 63376, US

(21) Appl. No.: 10/329,844

(22) Filed: Dec. 27, 2002

(51) Int. Cl. A61M 5/178

(52) U.S. Cl. 604/174

(57) ABSTRACT

The CADE (Catheter and delivery end) is a one piece intravenous IV that will allow the user to start a IV much easier then present technique. Three steps are needed at the present along with possible risk for needle stick and risk for blood contamination. The CADE is a one piece device not allowing for blood to be loss and minimizing the risk for needle stick.

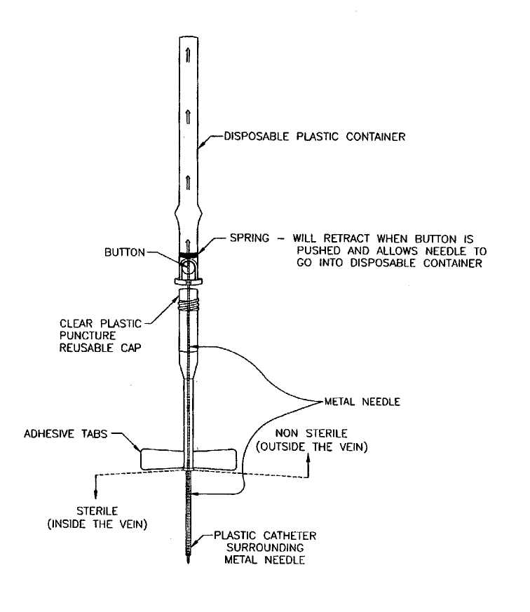

### CROSS REFERENCE

The CADE (Catheter and delivery end) is a one piece intravenous catheter. At the present a peripheral intravenous catheter is three steps or more and many pieces. I found three related topics that were most related to my idea. The first was the butterfly vacutainer with the patent filing number of #5120320. The second was a plastic cap that is screwed onto the catheter to close the system off with a reusable cap, it is called the Interlink System Injection Site with the U.S. Pat. Nos. D321,251; 5,167,648; 5,171,234; 5,188,620; Pat. Pending. The third device is called a Insyte Autoguard shielded I.V. Catheter. It is made by Becton Dickerson & Co. They have a main infusion division that is located at 9450 South State Street, Sandy Utah 84070. I have researched threw the United States Patent and Trademark office records and was unable to locate this device. I also tried to contact the company to obtain the Patent Number, but was unsuccessful at obtaining the number.

### BACKGROUND OF THE INVENTION

As a Registered Nurse I have started many IV's. I have seen many IV's and there has always been several problems. 1) The loss of blood upon initial insertion. 2) Keeping the IV in the vein in place while trying to tape the catheter down.

The loss of blood is due to the fact that after you insert an IV you have to either connect the open end of the catheter to IV tubing to receive some sort of fluids or to a cap to close off the IV. In the few seconds you are trying to connect your IV catheter you are trying to hold down the vein by placing a finger on top of the skin in front of the catheter so blood does not leak out. This rarely ever happens. Blood usually rushes out and ends up all over the patient and yourself. If you are successful in holding the vein down not allowing any blood to be lost you are trying to connect the open end of the catheter to tubing and usually end up loosing your initial IV site because you have not anchored it down yet with any tape.

I have thought long and hard before I came up with the solution. Have the IV all in one piece.

### BRIEF SUMMARY OF THE INVENTION

The CADE (Catheter and delivery end) is a one piece intravenous catheter. At the present a peripheral intravenous catheter is three steps or more and many pieces. First the actual catheter that is in the vein, secondly a cap or IV tubing of some sort to stop the blood flow from the open vein, third tape to place over the catheter after it is inserted. The CADE is a one piece device that does not allow blood to be exposed outside the body and can be easily adhered to the skin without loosing your IV site.

### BRIEF DESCRIPTION OF THE DRAWING

The drawing shows the catheter in two pieces. It will initially be a one piece device. After the needle is properly inserted into the vein the button will be pushed, the spring will release the metal needle, leaving the plastic catheter inside the vein then pushing the metal needle threw the reusable plastic end into the disposable container. This container will be able to pop off when the user has completed this step. The pop off plastic disposable container will have the contaminated metal needle inside and the user will be able to dispose of this in a biohazard friendly manner. This disposable container will not allow the user to be exposed to any needle sticks. The adhesive tabs will not only allow the user to help guide the catheter inside the vein easier but will also serve the purpose to help adhere the device in place. The needle and catheter that is beyond the tabs will be sterile since this will be left inside the body.

### DETAILED DESCRIPTION OF THE INVENTION

The CADE (Catheter and delivery end) is a one piece intravenous catheter. The CADE will allow the user to insert the IV into the vein using tabs to help guide the needle in smoothly. Once the visualization of a back flash of blood is present (the clear tubing leading to the reusable clear plastic end will show this) this is so the user will know they are successfully in a vein. Then the tabs can also serve the purpose to help hold/anchor the IV after it is properly inserted. The underneath portion of the tabs will have peel off plastic that will have an adhesive backing, thus allowing this to stick to the skin keeping the catheter in place. Once the IV is anchored you will push the button that will release the spring that will draw back the needle threw the reusable plastic end and into the plastic disposable container. This container will be able to snap off after the button is pushed and you will be able to dispose of the plastic container consisting of the contaminated needle properly into a biohazard sharps container. This will leave the plastic catheter inside the vein. The reusable plastic end will not only stop the flow of blood that usually occurs with the initial insertion of a peripheral IV but will allow additional needle sticks for future medications to pushed threw and for IV fluids to be connected. This will be a one step process that usually consist of no less then three separate pieces and will be more cost effective for the prospective customer.

1. I claim the CADE (catheter and delivery end), is a one piece intravenous catheter that will allow the user to easily start a IV by using the tabs to help guide the needle into the vein and also serve the purpose to adhere to the skin once the catheter is successfully inserted without the risk of blood loss R/T the reusable plastic end that will stop any blood loss after the initial insertion of the IV and also will allow for future needle sticks for IV fluids, IV medications.
